(19) United States
(12) Patent Application Publication
(10) Pub. No.: US 2025/0255639 A1
(43) Pub. Date: Aug. 14, 2025

(54) SYSTEM AND METHODS FOR MODIFYING VALVE LEAFLETS

(71) Applicant: BOSTON SCIENTIFIC SCIMED, INC., Maple Grove, MN (US)

(72) Inventors: John M. Edgell, Plymouth, MN (US); Levi Joel Wolterstorff, Saint Paul, MN (US); Todd Rowe, Excelsior, MN (US); James M. Anderson, Corcoran, MN (US); Lauren Koon, Mississauga (CA); Steven Kinio, Mississauga (CA)

(73) Assignee: BOSTON SCIENTIFIC SCIMED, INC., MAPLE GROVE, MN (US)

(21) Appl. No.: 19/052,781

(22) Filed: Feb. 13, 2025

Related U.S. Application Data
(60) Provisional application No. 63/553,253, filed on Feb. 14, 2024.

(51) Int. Cl. A61B 17/3205; A61B 18/00; A61B 18/14

(52) U.S. Cl. CPC A61B 17/32056; A61B 18/1492; A61B 2018/00369

(57) ABSTRACT

A system for modifying at least one valve leaflet of a heart valve may include an elongate shaft, an expandable balloon, and an expandable structure disposed radially outward of the balloon. In an expanded configuration, the expandable structure is configured to separate the at least one valve leaflet into discrete pieces. A system may include an expandable structure comprising an elongate finger and a clamping jaw disposed proximate a distal end of the finger. In an expanded configuration, the elongate finger is offset radially outward from the elongate shaft and is configured to separate the at least one valve leaflet into discrete pieces. A system may include an electrode disposed within the balloon and configured to energize the inflation fluid. In an inflated configuration, energized inflation fluid ejected through a plurality of small apertures in the balloon is configured to separate the at least one valve leaflet into discrete pieces.

### CROSS-REFERENCE TO RELATED APPLICATIONS

This application claims the benefit of priority of U.S. Provisional Application No. 63/553,253 filed Feb. 14, 2024, the entire disclosure of which is hereby incorporated by reference.

### TECHNICAL FIELD

The disclosure relates generally to medical devices and more particularly to medical devices that are adapted for modifying at least one valve leaflet of a heart valve (native or implanted).

### BACKGROUND

A wide variety of intracorporeal medical devices have been developed for medical use including medical devices for repair or replacement of diseased heart valves. Transcatheter valve replacement procedures rely on the replacement heart valve implant to push aside native valve leaflets (or replacement valve leaflets in instances where a second replacement heart valve implant is being implanted within a first replacement heart valve implant) in order to function properly. In some instances, diseased and/or fused valve leaflets may cause challenges for transcatheter valve replacement procedures. For example, the force required to push the diseased valve leaflets aside may exceed the capabilities of the replacement heart valve implant. In some instances, balloon valvuloplasty is performed prior to implanting the replacement heart valve implant to pre-dilate the heart valve. However, balloon valvuloplasty performed in conjunction with transcatheter valve replacement procedures may present its own complications and/or risks, and in some instances, stenosis or leaflet fusion may “survive” the balloon valvuloplasty procedure and prevent the replacement heart valve implant from fully expanding, properly seating within the valve annulus, and/or functioning properly. There is an ongoing need to provide alternative medical devices as well as alternative methods for manufacturing and using medical devices to improve the efficiency and success of transcatheter valve replacement procedures.

### SUMMARY

In one example, a system for modifying at least one valve leaflet of a heart valve may comprise an elongate shaft, an expandable balloon disposed proximate a distal end of the elongate shaft, wherein the expandable balloon may be configured to shift between a deflated configuration and an inflated configuration, and an expandable structure disposed radially outward of the expandable balloon, wherein the expandable structure may be configured to shift between a collapsed configuration and an expanded configuration. In the expanded configuration, the expandable structure may be configured to separate the at least one valve leaflet into a plurality of discrete pieces.0.23

In addition or alternatively to any example disclosed herein, the expandable structure comprises at least one elongate finger having a lumen extending therein.

In addition or alternatively to any example disclosed herein, a distal portion of each finger of the at least one elongate finger comprises a plurality of apertures formed therein in fluid communication with the lumen.

In addition or alternatively to any example disclosed herein, the plurality of apertures faces radially outward from the expandable balloon.

In addition or alternatively to any example disclosed herein, the lumen is in fluid communication with a source of pressurized fluid.

In addition or alternatively to any example disclosed herein, pressurized fluid ejected from the plurality of apertures is configured to separate the at least one valve leaflet into the plurality of discrete pieces.

In addition or alternatively to any example disclosed herein, the lumen is in fluid communication with a vacuum source.

In addition or alternatively to any example disclosed herein, a vacuum applied to the plurality of apertures is configured to draw a portion of the at least one valve leaflet into their respective finger to separate the at least one valve leaflet into the plurality of discrete pieces.

In addition or alternatively to any example disclosed herein, a system for modifying at least one valve leaflet of a heart valve may comprise an elongate shaft, and an expandable structure disposed at a distal end of the elongate shaft, the expandable structure comprising an elongate finger having a lumen extending therein and a clamping jaw disposed proximate a distal end of the elongate finger. The expandable structure may be configured to shift between a collapsed configuration and an expanded configuration. In the expanded configuration, the elongate finger is offset radially outward from the elongate shaft and may be configured to separate the at least one valve leaflet into a plurality of discrete pieces.

In addition or alternatively to any example disclosed herein, the clamping jaw is pivotably coupled to the elongate finger and configured to open radially inward toward a central longitudinal axis of the elongate shaft.

In addition or alternatively to any example disclosed herein, the elongate finger comprises a plurality of apertures formed therein in fluid communication with the lumen.

In addition or alternatively to any example disclosed herein, the lumen is in fluid communication with a source of pressurized fluid.

In addition or alternatively to any example disclosed herein, the lumen is in fluid communication with a vacuum source.

In addition or alternatively to any example disclosed herein, the plurality of apertures faces toward the clamping jaw.

In addition or alternatively to any example disclosed herein, the plurality of apertures is arranged linearly along the elongate finger.

In addition or alternatively to any example disclosed herein, the plurality of apertures is arranged along a perimeter of the elongate finger.

In addition or alternatively to any example disclosed herein, the clamping jaw is configured to cooperate with the elongate finger to grasp a portion of the at least one valve leaflet and remove the portion of at least one valve leaflet from the heart valve.

In addition or alternatively to any example disclosed herein, the expandable structure may further comprise a second elongate finger offset radially outward from the elongate shaft, the second elongate finger having a lumen extending therein and a second clamping jaw disposed proximate a distal end of the second elongate finger, and a third elongate finger offset radially outward from the elongate shaft, the third elongate finger having a lumen extending therein and a third clamping jaw disposed proximate a distal end of the third elongate finger.

In addition or alternatively to any example disclosed herein, a system for modifying at least one valve leaflet of a heart valve may comprise an elongate shaft, an expandable balloon disposed proximate a distal end of the elongate shaft, wherein the expandable balloon may be configured to shift between a deflated configuration and an inflated configuration via an inflation fluid, and an electrode disposed within the expandable balloon and in communication with the inflation fluid, wherein the electrode may be configured to energize the inflation fluid. The expandable balloon may comprise a proximal waist, a distal waist, and a body portion extending between the proximal waist and the distal waist. The body portion of the expandable balloon may comprise a plurality of small apertures in fluid communication with an interior of the body portion of the expandable balloon. In the inflated configuration, energized inflation fluid ejected through the plurality of small apertures may be configured to separate the at least one valve leaflet into a plurality of discrete pieces.

In addition or alternatively to any example disclosed herein, the elongate shaft comprises a marker element configured to align the plurality of small apertures with the at least one valve leaflet under fluoroscopy.

In addition or alternatively to any example disclosed herein, the plurality of small apertures comprises a first plurality of small apertures configured to separate a first valve leaflet into a first plurality of discrete pieces, and a second plurality of small apertures configured to separate a second valve leaflet into a second plurality of discrete pieces.

The above summary of some embodiments, aspects, and/or examples is not intended to describe each disclosed embodiment or every implementation of the present disclosure. The figures and detailed description which follow more particularly exemplify these embodiments.

### BRIEF DESCRIPTION OF THE DRAWINGS

The disclosure may be more completely understood in consideration of the following detailed description in connection with the accompanying drawings, in which:

FIG. 1 illustrates selected aspects of a system for modifying valve leaflets;

FIG. 2 illustrates selected aspects of a system for modifying valve leaflets;

FIG. 3 illustrates modified valve leaflets in accordance with the disclosure;

FIG. 4 illustrates selected aspects of a system for modifying valve leaflets;

FIG. 5 illustrates selected aspects of a system for modifying valve leaflets;

FIG. 6 illustrates selected aspects of a system for modifying valve leaflets;

FIGS. 7-8 illustrate selected aspects of a system for modifying valve leaflets;

FIGS. 9-10 illustrate selected aspects of a system for modifying valve leaflets;

FIG. 11 illustrates selected aspects related to the use of the system shown in FIGS. 7-8;

FIG. 12 illustrates selected aspects related to the use of the system shown in FIGS. 9-10;

FIG. 13 illustrates selected aspects of a system for modifying valve leaflets; and

FIGS. 14-15 illustrate selected aspects of a system for modifying valve leaflets.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to one feature may be equally referred to all instances and quantities beyond one of said feature unless clearly stated to the contrary. As such, it will be understood that the following discussion may apply equally to any and/or all components for which there are more than one within the device, etc. unless explicitly stated to the contrary.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently-such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to implement the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Additionally, it should be noted that in any given figure, some features may not be shown, or may be shown schematically, for clarity and/or simplicity. Additional details regarding some components and/or method steps may be illustrated in other figures in greater detail. The devices and/or methods disclosed herein may provide a number of desirable features and benefits as described in more detail below.

For the purpose of this disclosure, the discussion herein is directed toward treating a native heart valve such as the aortic valve and will be so described in the interest of brevity. This, however, is not intended to be limiting as the skilled person will recognize that the following discussion may also apply to other heart valves, and/or replacement heart valve implants, with no or minimal changes to the structure and/or scope of the disclosure.

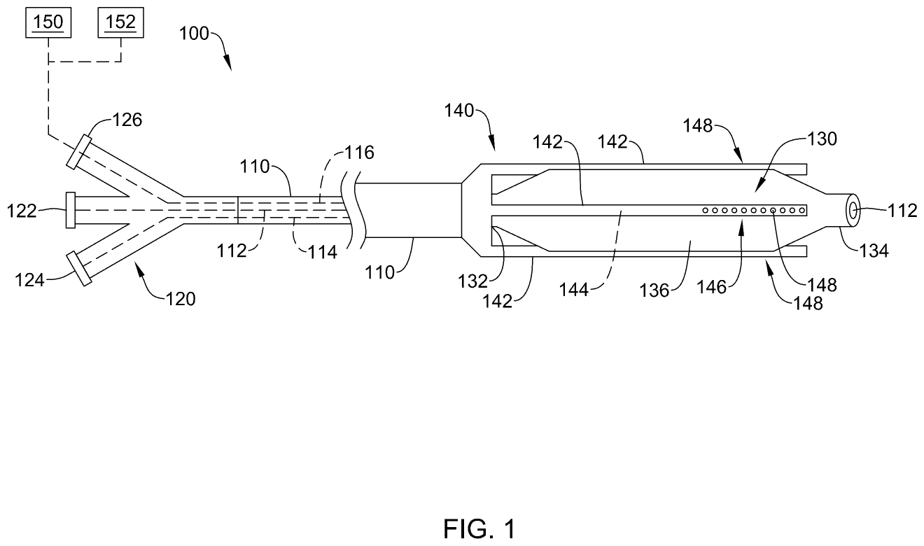

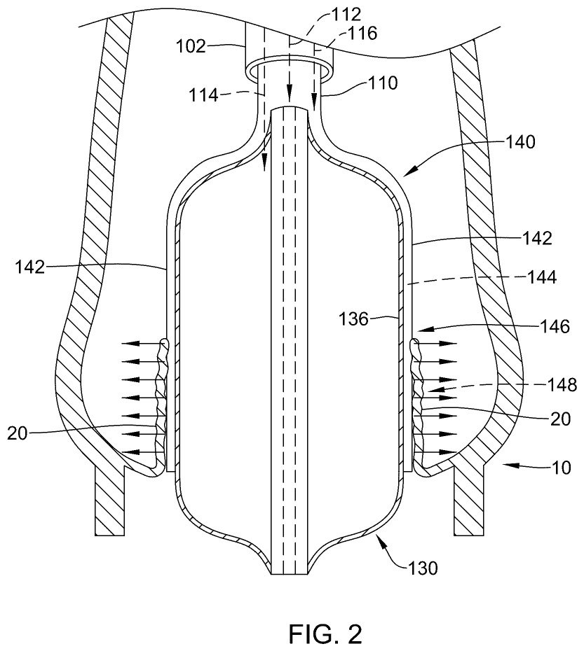

FIG. 1 illustrates selected aspects of a system 100 for modifying at least one valve leaflet 20 of a heart valve 10 (e.g., an aortic valve, a replacement heart valve implant, etc.), shown in partial cutaway view in FIGS. 2-4 for example. In some embodiments, the system 100 may comprise an elongate shaft 110. In some embodiments, the elongate shaft 110 may comprise a proximal manifold 120 disposed at a proximal end of the elongate shaft 110. In some embodiments, the proximal manifold 120 may comprise a plurality of ports. In some embodiments, the proximal manifold 120 may comprise a guidewire port 122 in communication with a guidewire lumen 112 extending through the elongate shaft 110 and/or to a distal end of the elongate shaft 110. In some embodiments, the guidewire lumen 112 may open distally at a distalmost end of the elongate shaft 110. Other configurations are also contemplated.

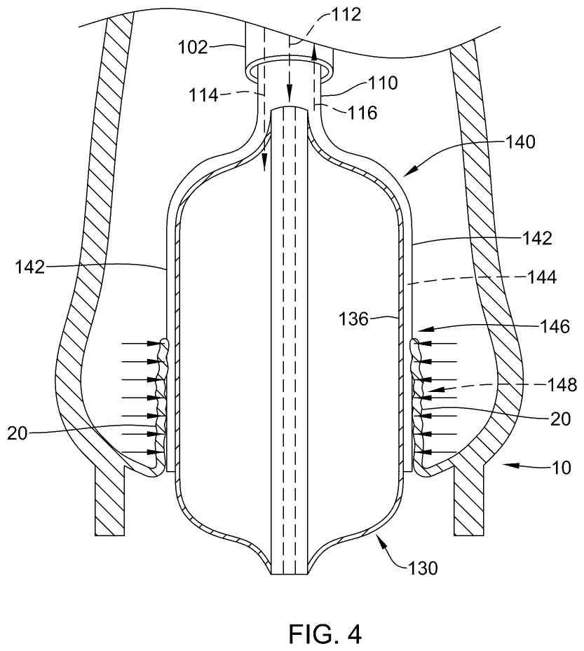

In some embodiments, the system 100 may comprise an expandable balloon 130 disposed proximate the distal end of the elongate shaft 110. In some embodiments, the expandable balloon 130 may be disposed immediately adjacent the distal end of the elongate shaft 110. In some embodiments, the expandable balloon 130 may be configured to shift between a deflated configuration (e.g., FIGS. 1, 5, 6) and an inflated configuration (e.g., FIGS. 2, 4) via an inflation fluid. In at least some embodiments, the expandable balloon 130 may comprise a proximal waist 132 fixedly attached to the elongate shaft 110, a distal waist 134 fixedly attached to the elongate shaft 110, and a body portion 136 extending between the proximal waist 132 and the distal waist 134. In some embodiments, the expandable balloon 130 may be formed from an elastic material. In some embodiments, the expandable balloon 130 may be formed from an inelastic material. In at least some embodiments, the expandable balloon 130 may be formed from a polymeric material. Other configurations are also contemplated.

In some embodiments, the proximal manifold 120 may comprise an inflation port 124 in fluid communication with an inflation lumen 114 extending from the inflation port 124 to the expandable balloon 130. In some embodiments, the inflation port 124 may be configured to be coupled to a source of inflation fluid. In some embodiments, the inflation fluid may be configured to receive and/or configured to accept an electrical charge.

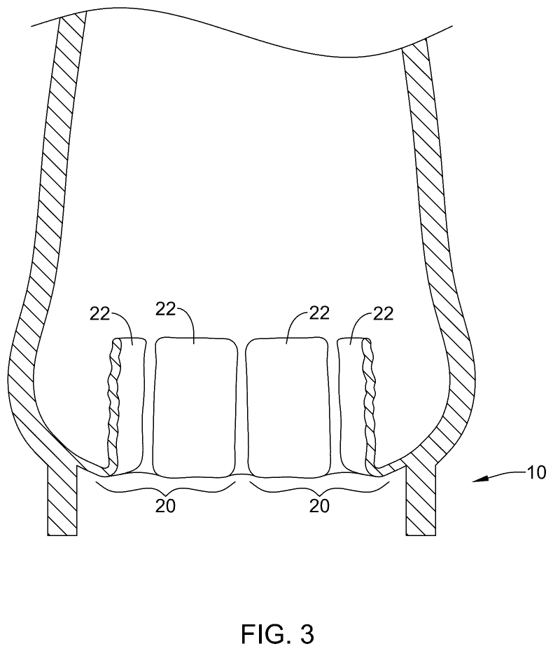

In some embodiments, the system 100 may comprise an expandable structure 140 disposed radially outward of the expandable balloon 130. In some embodiments, the expandable structure 140 may be disposed radially outward of the elongate shaft 110. In some embodiments, the expandable structure 140 may be configured to shift between a collapsed configuration (e.g., FIGS. 1, 5, 6) and an expanded configuration (e.g., FIGS. 2, 4). In some embodiments, in the expanded configuration, the expandable structure 140 may be configured to separate (e.g., cut, resect, divide, incise, split, etc.) the at least one valve leaflet 20 (e.g., FIGS. 2-4) of the heart valve 10 into a plurality of discrete pieces 22, as seen in FIG. 3 for example.

In some embodiments, the proximal manifold 120 may comprise a working port 126 in fluid communication with a working lumen 116 extending from the working port 126 to the expandable structure 140, and/or in fluid communication with the expandable structure 140. In some embodiments, the working port 126 may be configured to be coupled to a source of pressurized fluid 150. In some embodiments, the working port 126 may be configured to be coupled to a vacuum source 152. In some alternative embodiments, the working port 126 may be configured to be switchably and/or selectively coupled to the source of pressurized fluid 150 and the vacuum source 152.

The expandable structure 140 may comprise at least one elongate finger 142 having a lumen 144 extending therein. In some embodiments, the at least one elongate finger 142 may comprise a single finger. In some embodiments, the at least one elongate finger 142 may comprise a plurality of fingers (e.g., more than one finger). In the interest of brevity, the expandable structure 140 is illustrated with a plurality of fingers (e.g., FIG. 1), but the skilled person will readily understand that the scope of the disclosure does not require all of the illustrated fingers. In some embodiments, the at least one elongate finger 142 may comprise a first finger having a first lumen extending therein, a second finger having a second lumen extending therein, and a third finger having a third lumen extending therein. In some embodiments, such as embodiments comprising more than one finger and more than one lumen, each lumen may be fluid communication with a common lumen at and/or within a proximal portion of the expandable structure 140. The lumen 144 of the at least one elongate finger 142, and/or the first lumen of the first finger, the second lumen of the second finger, and the third lumen of the third finger, may be in fluid communication with the working lumen 116 of the elongate shaft 110. In some embodiments, the proximal portion of the expandable structure 140 may extend around a circumference of the elongate shaft 110. In some embodiments, the proximal portion of the expandable structure 140 may be fixedly attached to the elongate shaft 110.

In some embodiments, a distal portion 146 of each finger of the at least one elongate finger 142 may comprise a plurality of apertures 148 formed therein in fluid communication with the lumen 144 and/or the working lumen 116 of the elongate shaft 110. In some embodiments, a distal portion of the first finger may comprise a first plurality of apertures formed therein in fluid communication with the first lumen. In some embodiments, a distal portion of the second finger may comprise a second plurality of apertures formed therein in fluid communication with the second lumen. In some embodiments, a distal portion of the third finger may comprise a third plurality of apertures formed therein in fluid communication with the third lumen. In at least some embodiments, the plurality of apertures 148, and/or the first plurality of apertures, the second plurality of apertures, and the third plurality of apertures, may face radially outward and/or away from the expandable balloon 130. In some embodiments, the plurality of apertures 148, and/or the first plurality of apertures, the second plurality of apertures, and the third plurality of apertures, may face radially outward and/or away from a central longitudinal axis of the elongate shaft 110.

In some embodiments, the plurality of apertures 148, and/or the first plurality of apertures, the second plurality of apertures, and the third plurality of apertures, may be arranged in a substantially straight line or a linear pattern. In some embodiments, the plurality of apertures 148, and/or the first plurality of apertures, the second plurality of apertures, and the third plurality of apertures, may be arranged longitudinally and/or axially. In some embodiments, the plurality of apertures 148, and/or the first plurality of apertures, the second plurality of apertures, and the third plurality of apertures, may be arranged parallel with a central longitudinal axis of the elongate shaft 110. In some embodiments, the plurality of apertures 148, and/or the first plurality of apertures, the second plurality of apertures, and the third plurality of apertures, may be arranged in a non-linear pattern (e.g., a zig-zag shape, a curve, etc.). In some embodiments, the plurality of apertures 148, and/or the first plurality of apertures, the second plurality of apertures, and the third plurality of apertures, may be arranged in various patterns and/or configurations (e.g., different geometries, etc.).

In some embodiments, the at least one valve leaflet 20, and/or each leaflet of the at least one valve leaflet 20, may be cut and/or slit in a generally straight line. In embodiments, a portion or portions of the at least one valve leaflet 20, and/or each leaflet of the at least one valve leaflet 20, may be excised or removed entirely. Other configurations are also contemplated.

In some embodiments, the system 100 may comprise a delivery catheter 102, as seen in FIGS. 2 and 4. In some embodiments, the elongate shaft 110, the expandable balloon 130, and/or the expandable structure 140 may be axially slidable within a lumen of the delivery catheter 102. In some embodiments, the delivery catheter 102 may comprise an aspiration function and/or capability. In some embodiments, the system 100 may comprise a separate aspiration catheter delivered and/or extending alongside the elongate shaft 110. Other configurations are also contemplated. In some embodiments, the system 100 may be devoid of the delivery catheter 102.

In some embodiments, the expandable structure 140 may be secured to the elongate shaft 110. In some embodiments, the expandable structure 140 may be fixedly attached to the elongate shaft 110. In some embodiments, the expandable structure 140 may be secured and/or fixedly attached to the elongate shaft 110 via mechanical fasteners, adhesive bonding, welding, mechanical interference, friction fit, etc. In some embodiments, the expandable structure 140 may be integrally formed with and/or may be monolithically formed with the elongate shaft 110. Other configurations are also contemplated. In at least some embodiments, the expandable structure 140 may be formed from a metallic material. In some embodiments, the expandable structure 140 may be formed from a polymeric material. In some embodiments, the expandable structure 140 may be formed from a composite material. Other configurations are also contemplated.

In some embodiments, the expandable structure 140 may be formed from and/or may be integrally formed with an outer layer of the elongate shaft 110. In some embodiments, the expandable structure 140 may be formed from and/or may be integrally formed with an outermost layer of the elongate shaft 110. In some embodiments, the expandable structure 140 may be in fluid communication with an inner layer of the elongate shaft 110. Other configurations are also contemplated.

In some embodiments, the system 100 may be advanced to a position adjacent a heart valve 10 and/or a treatment site. In some embodiments, the expandable balloon 130 and the expandable structure 140 may be deployed from a distal end of the delivery catheter 102. In at least some embodiments, the expandable balloon 130 may be shifted from the deflated configuration (e.g., FIG. 1) toward and/or to the inflated configuration (e.g., FIGS. 2, 4) within the heart valve and/or the treatment site. In some embodiments, shifting the expandable balloon 130 from the deflated configuration toward and/or to the inflated configuration may shift the expandable structure 140 from the collapsed configuration toward and/or to the expanded configuration via an inflation fluid introduced through the inflation port 124. In some embodiments, one or more visualization means and/or methods may be used to align the at least one elongate finger 142 with the at least one valve leaflet 20 of the heart valve 10 before and/or during inflation of the expandable balloon 130 (e.g., shifting the expandable balloon 130 from the deflated configuration toward and/or to the inflated configuration). In some embodiments, the system 100 and/or the elongate shaft 110 may comprise a marker element (not shown) configured to align at least one elongate finger 142 with the at least one valve leaflet 20 of the heart valve 10 before and/or during inflation of the expandable balloon 130 (e.g., shifting the expandable balloon 130 from the deflated configuration toward and/or to the inflated configuration). In some alternative configurations, the expandable structure 140 may be shifted from the collapsed configuration toward and/or to the expanded configuration without shifting the expandable balloon 130 from the deflated configuration toward and/or to the inflated configuration. Other configurations and/or means of aligning and/or orienting the system 100 are also contemplated.

In some embodiments, shifting the expandable balloon 130 from the deflated configuration toward and/or to the inflated configuration may deflect and/or urge the at least one valve leaflet 20, and/or each valve leaflet of the at least one valve leaflet 20, toward an open position wherein the at least one valve leaflet 20, and/or each valve leaflet of the at least one valve leaflet 20, extends downstream from an annulus of the heart valve 10. The plurality of apertures 148, and/or the first plurality of apertures, the second plurality of apertures, and the third plurality of apertures, may be substantially aligned with the at least one valve leaflet 20, and/or each valve leaflet of the at least one valve leaflet 20.

In some embodiments, pressurized fluid introduced into the working lumen 116 through the working port 126 may flow from the proximal manifold 120 to and/or out of the plurality of apertures 148, and/or the first plurality of apertures, the second plurality of apertures, and the third plurality of apertures. In some embodiments, the plurality of apertures 148, and/or the first plurality of apertures, the second plurality of apertures, and the third plurality of apertures, may form and/or direct the pressurized fluid into fluid jets directed at the at least one valve leaflet 20, and/or each valve leaflet of the at least one valve leaflet 20. In some embodiments, the pressurized fluid may be water, saline solution, or another biocompatible fluid. In at least some embodiments, the pressurized fluid may have a salinity level configured to conduct electrical energy and/or suitable for conducting electrical energy. In some embodiments, the pressurized fluid may include and/or may be a solution, a suspension, and/or may include one or more conduction enhancing elements added thereto. In some embodiments, the one or more conduction enhancing elements may be electrically conductive or electrically semi-conductive. Some suitable but non-limiting examples for the one or more conduction enhancing elements may include graphene, carbon, metallic materials, silicates, etc. In some embodiments, pressurized fluid ejected from the plurality of apertures 148, and/or the first plurality of apertures, the second plurality of apertures, and the third plurality of apertures, as seen in FIG. 2, may be configured to separate (e.g., cut, resect, divide, incise, split, etc.) the at least one valve leaflet 20, and/or each valve leaflet of the at least one valve leaflet 20 into the plurality of discrete pieces 22, as seen in FIG. 3.

In some embodiments, the pressurized fluid may be pressurized to at least 5000 pounds per square inch (psi) or at least about 34,400 kilopascals (kPa). In some embodiments, the pressurized fluid may be pressurized to at least 10,000 pounds per square inch (psi) or at least about 68,900 kilopascals (kPa). In some embodiments, the pressurized fluid may be pressurized to at least 15,000 pounds per square inch (psi) or at least about 103,000 kilopascals (kPa). The expandable structure 140 and/or the working lumen 116 may be adapted, configured, and designed with characteristics that enable the expandable structure 140 and/or the working lumen 116 to withstand and/or utilize high pressure fluid(s) without failure. In some embodiments, the system 100 may comprise a shield (not shown) disposed between the at least one valve leaflet 20, and/or each valve leaflet of the at least one valve leaflet 20, and surrounding tissue(s) to prevent and/or limit damage to the surrounding tissue(s). Other configurations are also contemplated.

In some embodiments, suction and/or a vacuum applied to the working lumen 116 through the working port 126 may draw and/or suck the at least one valve leaflet 20, and/or each valve leaflet of the at least one valve leaflet 20, against the at least one elongate finger 142, and/or the first finger, the second finger, and the third finger. In some embodiments, suction and/or a vacuum applied to the working lumen 116 through the working port 126 may draw and/or suck the at least one valve leaflet 20, and/or each valve leaflet of the at least one valve leaflet 20, into the plurality of apertures 148, and/or the first plurality of apertures, the second plurality of apertures, and the third plurality of apertures. In some embodiments, suction and/or a vacuum applied to the working lumen 116 through the working port 126 may draw and/or suck small volumes of leaflet tissue and/or stenosis into the plurality of apertures 148, and/or the first plurality of apertures, the second plurality of apertures, and the third plurality of apertures.

In some embodiments, suction and/or a vacuum applied to the plurality of apertures 148, and/or the first plurality of apertures, the second plurality of apertures, and the third plurality of apertures, may be configured to draw and/or suck a portion of the at least one valve leaflet 20, and/or each valve leaflet of the at least one valve leaflet 20, into their respective finger of the at least one elongate finger 142, and/or the first finger, the second finger, and the third finger, to separate (e.g., cut, resect, divide, incise, split, etc.) the at least one valve leaflet 20, and/or each valve leaflet of the at least one valve leaflet 20 into the plurality of discrete pieces 22.

In some embodiments, suction and/or a vacuum applied to the working lumen 116 through the working port 126, and/or to the plurality of apertures 148, and/or the first plurality of apertures, the second plurality of apertures, and the third plurality of apertures, as seen in FIG. 4, may be configured to separate (e.g., cut, resect, divide, incise, split, etc.) the at least one valve leaflet 20, and/or each valve leaflet of the at least one valve leaflet 20 into the plurality of discrete pieces 22, as seen in FIG. 3. In some embodiments, irrigation fluid may be supplied to the heart valve and/or the treatment site outside of the expandable structure 140, such as via the elongate shaft 110, the delivery catheter 102, or another means. As small volumes of leaflet tissue and/or stenosis are pulled and/or sucked into the plurality of apertures 148, and/or the first plurality of apertures, the second plurality of apertures, and the third plurality of apertures, the irrigation fluid may be suctioned away from the at least one elongate finger 142, and/or the first finger, the second finger, and the third finger, through the working lumen 116, thereby carrying the small volume of leaflet tissue and/or stenosis out of the heart valve 10 and/or the patient.

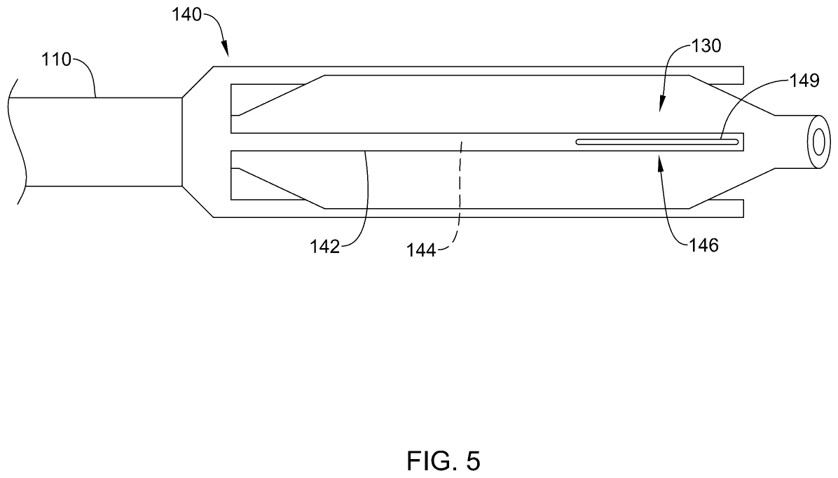

FIG. 5 illustrates selected aspects of an alternative configuration for the expandable structure 140 of FIGS. 1-4. Similar to above, the expandable structure 140 may comprise at least one elongate finger 142. In some embodiments, the at least one elongate finger 142 may comprise a single finger. In some embodiments, the at least one elongate finger 142 may comprise a plurality of fingers (e.g., more than one finger). In the interest of brevity, the expandable structure 140 is illustrated with a plurality of fingers (e.g., FIG. 5), but the skilled person will readily understand that the scope of the disclosure does not require all of the illustrated fingers.

In some embodiments, the distal portion 146 of the at least one elongate finger 142, the distal portion 146 of each finger of the at least one elongate finger 142, and/or the distal portion of the first finger, the distal portion of the second finger, and the distal portion of the third finger, may comprise a narrow slot 149 extending along the distal portion 146 in place of the plurality of apertures 148 (e.g., FIG. 1) described herein. Similar to the plurality of apertures 148, the narrow slot 149 may be in fluid communication with the lumen 144 of the at least one elongate finger 142, and/or the first lumen of the first finger, the second lumen of the second finger, and the third lumen of the third finger, as well as the working lumen 116 of the elongate shaft 110.

In some embodiments, a distal portion of the first finger may comprise a first narrow slot formed therein in fluid communication with the first lumen. In some embodiments, a distal portion of the second finger may comprise a second narrow slot formed therein in fluid communication with the second lumen. In some embodiments, a distal portion of the third finger may comprise a third narrow slot formed therein in fluid communication with the third lumen. In at least some embodiments, the narrow slot 149, and/or the first narrow slot, the second narrow slot, and the third narrow slot, may face radially outward and/or away from the expandable balloon 130. In some embodiments, the narrow slot 149, and/or the first narrow slot, the second narrow slot, and the third narrow slot, may face radially outward and/or away from a central longitudinal axis of the elongate shaft 110.

In some embodiments, the narrow slot 149, and/or the first narrow slot, the second narrow slot, and the third narrow slot, may be arranged in a substantially straight line or linearly. In some embodiments, the narrow slot 149, and/or the first narrow slot, the second narrow slot, and the third narrow slot, may be arranged longitudinally and/or axially. In some embodiments, the narrow slot 149, and/or the first narrow slot, the second narrow slot, and the third narrow slot, may be arranged parallel with a central longitudinal axis of the elongate shaft 110. In some embodiments, the narrow slot 149, and/or the first narrow slot, the second narrow slot, and the third narrow slot, may be arranged non-linearly (e.g., a zig-zag shape, a curve, etc.). In some embodiments, the narrow slot 149, and/or the first narrow slot, the second narrow slot, and the third narrow slot, may be arranged in various shapes and/or configurations (e.g., different geometries, etc.).

In some embodiments, the narrow slot 149, and/or the first narrow slot, the second narrow slot, and the third narrow slot, may be configured to separate (e.g., cut, resect, divide, incise, split, etc.) the at least one valve leaflet 20 of the heart valve 10 into a plurality of discrete pieces 22, as seen in FIG. 3. In some embodiments, the narrow slot 149, and/or the first narrow slot, the second narrow slot, and the third narrow slot, may be configured to cooperate with and/or to work with the source of pressurized fluid and/or the vacuum source in a manner similar to the plurality of apertures 148 described herein.

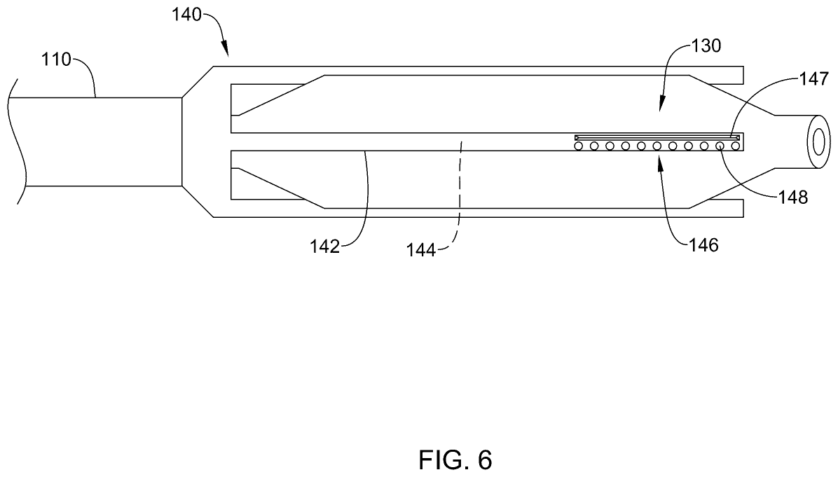

FIG. 6 illustrates selected aspects of an alternative configuration for the expandable structure 140 of FIGS. 1-4. Similar to above, the expandable structure 140 may comprise at least one elongate finger 142. In some embodiments, the at least one elongate finger 142 may comprise a single finger. In some embodiments, the at least one elongate finger 142 may comprise a plurality of fingers (e.g., more than one finger). In the interest of brevity, the expandable structure 140 is illustrated with a plurality of fingers (e.g., FIG. 5), but the skilled person will readily understand that the scope of the disclosure does not require all of the illustrated fingers.

In some embodiments, the distal portion 146 of the at least one elongate finger 142, the distal portion 146 of each finger of the at least one elongate finger 142, and/or the distal portion of the first finger, the distal portion of the second finger, and the distal portion of the third finger, may comprise the plurality of apertures 148 extending along the distal portion 146 as described herein. Additionally, the distal portion 146 of the at least one elongate finger 142, the distal portion 146 of each finger of the at least one elongate finger 142, and/or the distal portion of the first finger, the distal portion of the second finger, and the distal portion of the third finger, may comprise a cutting blade 147 extending alongside the plurality of apertures 148. In some embodiments, the first finger may comprise a first cutting blade extending alongside the first plurality of apertures, the second finger may comprise a second cutting blade extending alongside the second plurality of apertures, and the third finger may comprise a third cutting blade extending alongside the third plurality of apertures.

In some embodiments, the plurality of apertures 148 and the cutting blade 147, and/or the first plurality of apertures and the first cutting blade, the second plurality of apertures and the second cutting blade, and the third plurality of apertures and the third cutting blade, may be arranged in a substantially straight line or linearly. In some embodiments, the plurality of apertures 148 and the cutting blade 147, and/or the first plurality of apertures and the first cutting blade, the second plurality of apertures and the second cutting blade, and the third plurality of apertures and the third cutting blade, may be arranged longitudinally and/or axially. In some embodiments, the plurality of apertures 148 and the cutting blade 147, and/or the first plurality of apertures and the first cutting blade, the second plurality of apertures and the second cutting blade, and the third plurality of apertures and the third cutting blade, may be arranged parallel with a central longitudinal axis of the elongate shaft 110. In some embodiments, the plurality of apertures 148 and the cutting blade 147, and/or the first plurality of apertures and the first cutting blade, the second plurality of apertures and the second cutting blade, and the third plurality of apertures and the third cutting blade, may be arranged non-linearly (e.g., a zig-zag shape, a curve, etc.). In some embodiments, the plurality of apertures 148 and the cutting blade 147, and/or the first plurality of apertures and the first cutting blade, the second plurality of apertures and the second cutting blade, and the third plurality of apertures and the third cutting blade, may be arranged in various shapes and/or configurations (e.g., different geometries, etc.). While the cutting blade(s) of the disclosure is generally arranged and/or positioned alongside and/or generally parallel to the immediately adjacent plurality of apertures, the skilled person will recognize that this may not be required and that other arrangements are also contemplated and/or may also be useful.

In some embodiments, the plurality of apertures 148, and/or the first plurality of apertures, the second plurality of apertures, and the third plurality of apertures, in cooperation with the cutting blade 147, and/or the first cutting blade, the second cutting blade, and the third cutting blade, may be configured to separate (e.g., cut, resect, divide, incise, split, etc.) the at least one valve leaflet 20 of the heart valve 10 into a plurality of discrete pieces 22, when suction and/or a vacuum is applied to the working lumen 116 through the working port 126, and/or when suction and/or the vacuum is applied to the plurality of apertures 148, and/or the first plurality of apertures, the second plurality of apertures, and the third plurality of apertures, similar to FIG. 4.

In at least some embodiments, when suction and/or a vacuum is applied to the working lumen 116 through the working port 126, and/or when suction and/or the vacuum is applied to the plurality of apertures 148, and/or the first plurality of apertures, the second plurality of apertures, and the third plurality of apertures, a portion of the at least one valve leaflet 20, and/or a portion of each valve leaflet of the at least one valve leaflet 20, may be drawn and/or pulled against the cutting blade 147, and/or the first cutting blade, the second cutting blade, and the third cutting blade, to separate (e.g., cut, resect, divide, incise, split, etc.) the at least one valve leaflet 20, and/or each valve leaflet of the at least one valve leaflet 20 into the plurality of discrete pieces 22. In some embodiments, the suction and/or the vacuum applied to the working lumen 116 through the working port 126, to the plurality of apertures 148, and/or the first plurality of apertures, the second plurality of apertures, and the third plurality of apertures, may be insufficient to draw and/or suck the portion of the at least one valve leaflet 20, and/or the portion of each valve leaflet of the at least one valve leaflet 20, into the plurality of apertures 148 and/or the lumen 144. As such, only the cutting blade 147, and/or the first cutting blade, the second cutting blade, and the third cutting blade, physically separates (e.g., cuts, resects, divides, incises, splits, etc.) the at least one valve leaflet 20, and/or each valve leaflet of the at least one valve leaflet 20 into the plurality of discrete pieces 22.

In some embodiments, when suction and/or the vacuum is applied to the working lumen 116 through the working port 126, and/or when suction and/or the vacuum is applied to the plurality of apertures 148, and/or the first plurality of apertures, the second plurality of apertures, and the third plurality of apertures, the suction and/or the vacuum may be configured to draw and/or suck the portion of the at least one valve leaflet 20, and/or the portion of each valve leaflet of the at least one valve leaflet 20, into their respective finger of the at least one elongate finger 142, and/or the first finger, the second finger, and the third finger, and against the cutting blade 147, and/or the first cutting blade, the second cutting blade, and the third cutting blade, to separate (e.g., cut, resect, divide, incise, split, etc.) the at least one valve leaflet 20, and/or each valve leaflet of the at least one valve leaflet 20 into the plurality of discrete pieces 22. As such, both the plurality of apertures 148, and/or the first plurality of apertures, the second plurality of apertures, and the third plurality of apertures, and the cutting blade 147, and/or the first cutting blade, the second cutting blade, and the third cutting blade, physically separate (e.g., cut, resect, divide, incise, split, etc.) the at least one valve leaflet 20, and/or each valve leaflet of the at least one valve leaflet 20 into the plurality of discrete pieces 22.

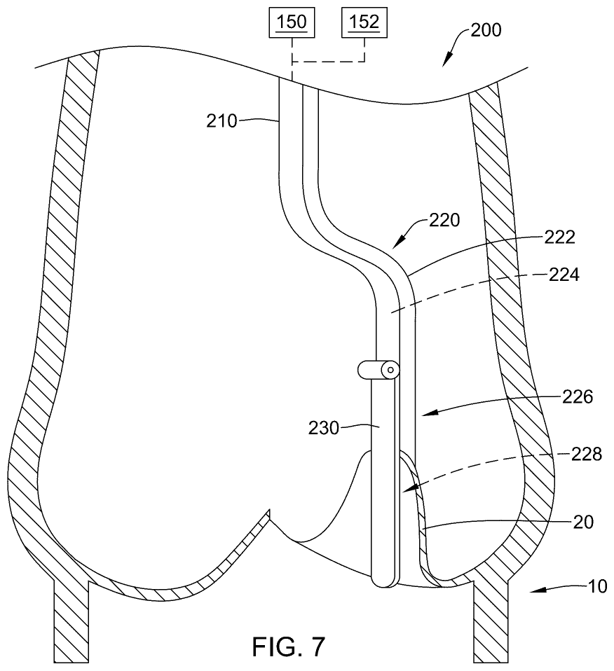

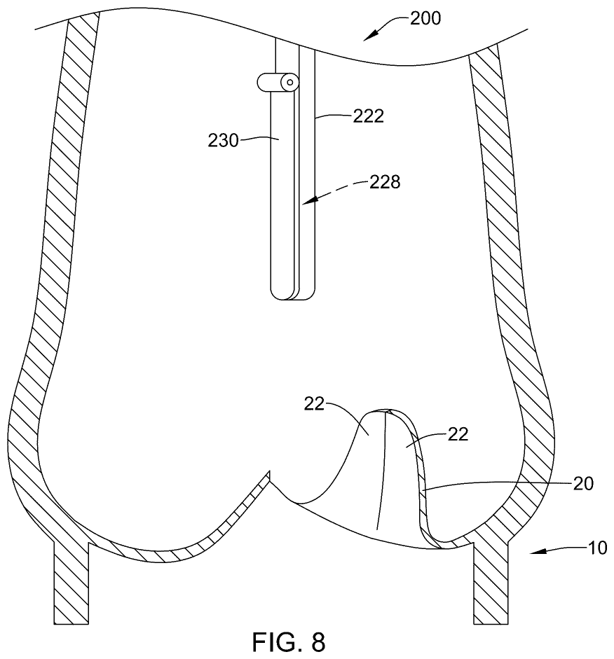

FIGS. 7-8 illustrate selected aspects of a system 200 for modifying at least one valve leaflet 20 of a heart valve 10 (e.g., an aortic valve, a replacement heart valve implant, etc.). In some embodiments, the system 200 may comprise an elongate shaft 210. In some embodiments, the elongate shaft 210 may comprise a proximal handle (not shown) disposed at a proximal end of the elongate shaft 210. In some embodiments, the proximal handle may comprise a guidewire port in communication with a guidewire lumen extending through the elongate shaft 210. In some embodiments, the proximal handle may comprise an actuation mechanism (not shown). In some embodiments, the actuation mechanism may comprise a knob, a button, a slider, etc. In some embodiments, the proximal handle may comprise a working port in fluid communication with a working lumen extending within and/or through the elongate shaft 210. In at least some embodiments, the working lumen may be separate and distinct from the guidewire lumen. In some embodiments, the working lumen may be spaced apart from the guidewire lumen.

In some embodiments, the system 200 may comprise an expandable structure 220 disposed at a distal end of the elongate shaft 210. In some embodiments, the expandable structure 220 may be fixedly attached to the elongate shaft 210 and/or the distal end of the elongate shaft 210. In some embodiments, the expandable structure 220 may be integrally formed and/or monolithically formed with the elongate shaft 210. Other configurations are also contemplated.

In some embodiments, the system 200 may comprise a delivery catheter (e.g., similar to the delivery catheter 102 described above). In some embodiments, the elongate shaft 210 and/or the expandable structure 220 may be axially slidable within a lumen of the delivery catheter. In some embodiments, the delivery catheter may comprise an aspiration function and/or capability. In some embodiments, the system 200 may comprise a separate aspiration catheter delivered and/or extending alongside the elongate shaft 210. Other configurations are also contemplated. In some embodiments, the system 200 may be devoid of the delivery catheter.

In some embodiments, the expandable structure 220 may comprise an elongate finger 222 having a lumen 224 extending therein. In some embodiments, the lumen 224 of the elongate finger 222 may be in fluid communication with the working lumen and/or the working port. In some embodiments, the working port may be configured to be coupled to a source of pressurized fluid 150. In some embodiments, the working port may be configured to be coupled to a vacuum source 152. In some alternative embodiments, the working port may be configured to be switchably and/or selectively coupled to the source of pressurized fluid 150 and the vacuum source 152.

In some embodiments, at least a portion of the expandable structure 220 may be disposed radially outward of the elongate shaft 210. In some embodiments, the expandable structure 220 and/or the elongate finger 222 may be configured to shift between a collapsed configuration (e.g., FIG. 8) or a straightened configuration, and an expanded configuration (e.g., FIG. 7) or an offset configuration. In some embodiments, in the expanded configuration or the offset configuration, the expandable structure 220 and/or the elongate finger 222 may be offset radially outward from and/or relative to the elongate shaft 210 and/or a central longitudinal axis of the elongate shaft 210. In some embodiments, in the expanded configuration or the offset configuration, the expandable structure 220 and/or the elongate finger 222 may be configured to separate (e.g., cut, resect, divide, incise, split, etc.) the at least one valve leaflet 20 of the heart valve 10 into a plurality of discrete pieces 22 (e.g., FIG. 8).

In some embodiments, the expandable structure 220 and/or the elongate finger 222 may be configured to be rotated about a central longitudinal axis of the elongate shaft 210 and/or repositioned (e.g., rotated, etc.) within the heart valve 10 to engage and/or treat other and/or different valve leaflets of the heart valve 10 (e.g., one valve leaflet at a time).

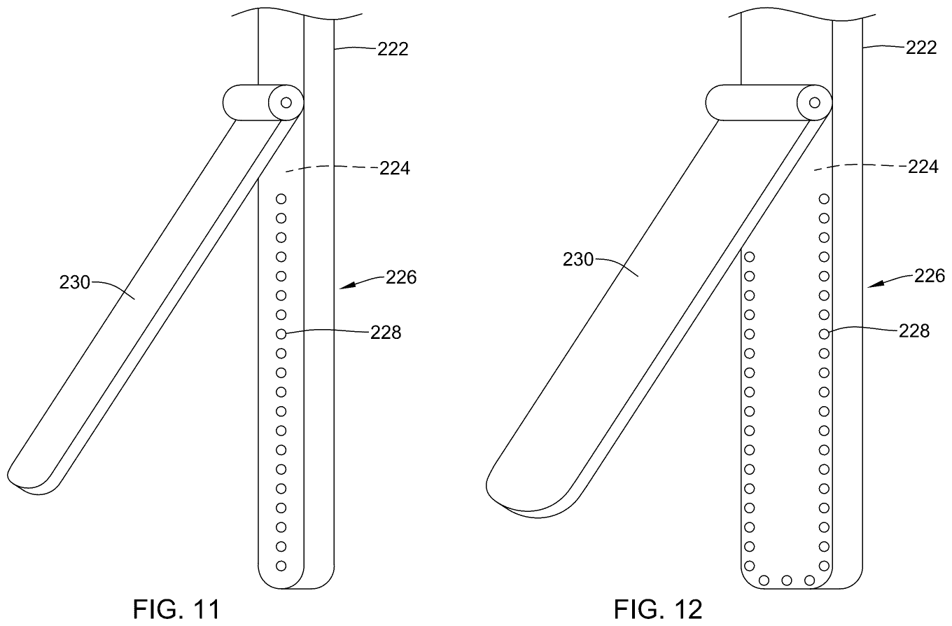

In some embodiments, the expandable structure 220 and/or the elongate finger 222 may comprise a clamping jaw 230 disposed proximate a distal end of the elongate finger 222. The clamping jaw 230 may be configured to shift between a closed position (e.g., FIGS. 7-8) and an open position (e.g., FIG. 11). In some embodiments, the clamping jaw 230 may be configured to engage with a distal portion 226 of the elongate finger 222 in the closed position. The clamping jaw 230 may be configured to grasp, clamp, pinch, hold, etc. the at least one valve leaflet 20 of the heart valve 10 in the closed position.

In some embodiments, the clamping jaw 230 may be pivotably coupled and/or pivotably attached to the elongate finger 222 and/or the distal portion 226 of the elongate finger 222. In some embodiments, the clamping jaw 230 may be configured to open radially inward from the distal portion 226 and/or the distal end of the elongate finger 222. In some embodiments, the clamping jaw 230 may be configured to open radially inward toward the central longitudinal axis of the elongate shaft 210. In some embodiments, the clamping jaw 230 may be configured to open radially inward when shifting from the closed position (e.g., FIGS. 7-8) toward and/or to the open position (e.g., FIG. 11). Other configurations are also contemplated.

In some embodiments, the elongate finger 222 may comprise a plurality of apertures 228 formed therein. In some embodiments, the plurality of apertures 228 may be in fluid communication with the lumen 224 of the elongate finger 222 and/or the working lumen and/or the working port. In at least some embodiments, the plurality of apertures 228 may face toward the clamping jaw 230. In some embodiments, the plurality of apertures 228 may face toward the central longitudinal axis of the elongate shaft 210. In some embodiments, the plurality of apertures 228 may face toward the central longitudinal axis of the elongate shaft 210 in the expanded configuration or the offset configuration.

In some embodiments, the plurality of apertures 228, the lumen 224, the working lumen, and/or the working port may be in fluid communication with the source of pressurized fluid 150. In some embodiments, the plurality of apertures 228, the lumen 224, the working lumen, and/or the working port may be in fluid communication with the vacuum source 152.

In some embodiments, pressurized fluid introduced into the working lumen through the working port may flow to and/or out of the plurality of apertures 228. In some embodiments, the plurality of apertures 228 may form and/or direct the pressurized fluid into fluid jets directed at the at least one valve leaflet 20. In some embodiments, the pressurized fluid may be water, saline solution, or another biocompatible fluid. In some embodiments, pressurized fluid ejected from the plurality of apertures 228 may be configured to separate (e.g., cut, resect, divide, incise, split, etc.) the at least one valve leaflet 20 into the plurality of discrete pieces 22, as seen in FIG. 8.

In some embodiments, the pressurized fluid may be pressurized to at least 5000 pounds per square inch (psi) or at least about 34,400 kilopascals (kPa). In some embodiments, the pressurized fluid may be pressurized to at least 10,000 pounds per square inch (psi) or at least about 68,900 kilopascals (kPa). In some embodiments, the pressurized fluid may be pressurized to at least 15,000 pounds per square inch (psi) or at least about 103,000 kilopascals (kPa). The expandable structure 220 and/or the working lumen may be adapted, configured, and designed with characteristics that enable the expandable structure 220 and/or the working lumen to withstand and/or utilize high pressure fluid(s) without failure.

In some embodiments, the clamping jaw 230 may form and/or function as a shield or a backstop for the pressurized fluid ejected from the plurality of apertures 228 to facilitate separation of the at least one valve leaflet 20 into the plurality of discrete pieces 22 and/or to prevent and/or limit damage to the surrounding tissue(s). Other configurations are also contemplated. In some alternative configurations, the clamping jaw 230 may comprise at least one opening (not shown) aligned with the plurality of apertures 228 such that the pressurized fluid may pass completely through the at least one valve leaflet 20 and the clamping jaw 230. In some embodiments, the at least one opening may comprise a single opening (e.g., a slot, etc.) aligned with the plurality of apertures 228 such that each aperture of the plurality of apertures 228 is directed toward the single opening. In some embodiments, the at least one opening may comprise a plurality of openings aligned with the plurality of apertures 228 such that each aperture of the plurality of apertures 228 is directed toward one opening of the plurality of openings. Other configurations, including combinations thereof, are also contemplated.

In some embodiments, suction and/or a vacuum applied to the working lumen through the working port may draw and/or suck the at least one valve leaflet 20 against the elongate finger 222. In some embodiments, suction and/or a vacuum applied to the working lumen through the working port may draw and/or suck the at least one valve leaflet 20 into the plurality of apertures 228. In some embodiments, suction and/or a vacuum applied to the working lumen through the working port may draw and/or suck small volumes of leaflet tissue and/or stenosis into the plurality of apertures 228 and/or the elongate finger 222. In some embodiments, suction and/or a vacuum applied to the plurality of apertures 228 may be configured to draw and/or suck a portion of the at least one valve leaflet 20 into the elongate finger 222 to separate (e.g., cut, resect, divide, incise, split, etc.) the at least one valve leaflet 20 into the plurality of discrete pieces 22.

In some embodiments, suction and/or a vacuum applied to the working lumen through the working port, and/or to the plurality of apertures 228 may be configured to separate (e.g., cut, resect, divide, incise, split, etc.) the at least one valve leaflet 20 into the plurality of discrete pieces 22, as seen in FIG. 8. In some embodiments, irrigation fluid may be supplied to the heart valve and/or the treatment site outside of the expandable structure 220, such as via the elongate shaft 210, the delivery catheter, or another means. As small volumes of leaflet tissue and/or stenosis are pulled and/or sucked into the plurality of apertures 228, the irrigation fluid may be suctioned away from the elongate finger 222, through the lumen 224, and/or through the working lumen, thereby carrying the small volume of leaflet tissue and/or stenosis out of the heart valve 10 and/or the patient.

In some embodiments, the clamping jaw 230 may cooperate with the at least one valve leaflet 20 to form a seal around the plurality of apertures 228 to facilitate separation of the at least one valve leaflet 20 into the plurality of discrete pieces 22. In some alternative configurations, the clamping jaw 230 may comprise at least one opening (not shown) aligned with the plurality of apertures 228 such that the suction and/or the vacuum may pull irrigation fluid and/or blood through the at least one opening and the clamping jaw 230 to facilitate aspiration of the small volumes of leaflet tissue and/or stenosis through the lumen 224. In some embodiments, the at least one opening may comprise a single opening (e.g., a slot, etc.) aligned with the plurality of apertures 228 such that each aperture of the plurality of apertures 228 is aligned with the single opening. In some embodiments, the at least one opening may comprise a plurality of openings aligned with the plurality of apertures 228 such that each aperture of the plurality of apertures 228 is aligned with one opening of the plurality of openings. Other configurations, including combinations thereof, are also contemplated.

In some embodiments, the plurality of apertures 228 may be arranged in a substantially straight line or a linear pattern along the elongate finger 222, as shown in FIG. 11. In some embodiments, the plurality of apertures 228 may be arranged linearly along the elongate finger 222. In some embodiments, the plurality of apertures 228 may be arranged longitudinally and/or axially along the elongate finger 222. In some embodiments, the plurality of apertures 228 may be arranged along the elongate finger 222 parallel with a central longitudinal axis of the elongate shaft 210. In some embodiments, the at least one valve leaflet 20 may be cut and/or slit in a generally straight line.

In some embodiments, the plurality of apertures 228 may be arranged in a non-linear pattern (e.g., a zig-zag shape, a curve, etc.) along the elongate finger 222. In some embodiments, the plurality of apertures 228 may be arranged in various patterns, shapes, and/or configurations (e.g., different geometries, etc.) along the elongate finger 222. In some embodiments, the at least one valve leaflet 20 may be cut and/or slit in a non-straight line.

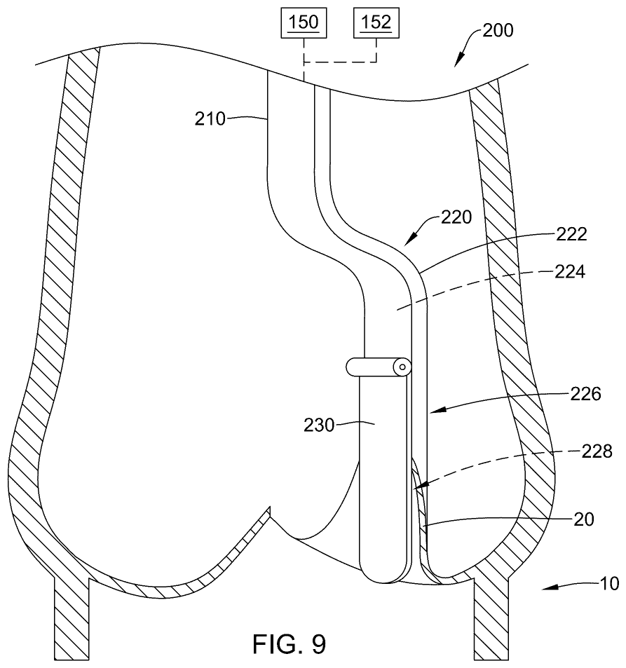

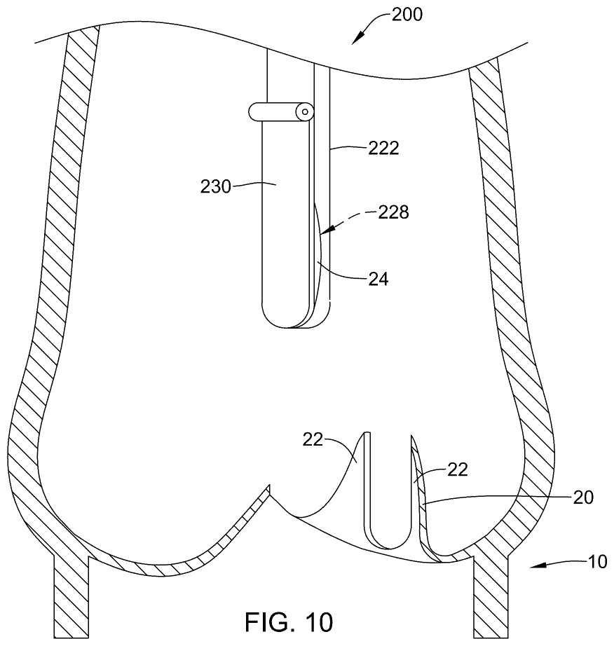

In some embodiments, at least the distal portion 226 of the elongate finger 222 may have an increased width, an increased size, an increased square area, etc., as seen schematically in FIGS. 9-10, compared to the configuration shown in FIGS. 7-8. That is, the distal portion 226 of the elongate finger 222 of FIGS. 9-10 may be “bigger” and/or shaped differently than the distal portion 226 of the elongate finger 222 of FIGS. 7-8. In some embodiments, the distal portion 226 of the elongate finger 222 may have a teardrop shape, a paddle shape, a trapezoidal shape, a triangular shape, etc. wherein a distalmost portion of the distal portion 226 has a greater circumferential extent than a proximal portion of the elongate finger 222. In some embodiments, an entire length of the elongate finger 222 may have the same width and/or circumferential extent. Other configurations are also contemplated.

In some embodiments, the plurality of apertures 228 may be arranged along a perimeter of the elongate finger 222, as seen in FIG. 12. In embodiments, a portion 24 of the at least one valve leaflet 20 may be excised or removed entirely, as seen in FIG. 10. In some embodiments, the clamping jaw 230 may be configured to cooperate with the elongate finger 222 to grasp, clamp, pinch, hold, etc. a portion 24 of the at least one valve leaflet 20 of the heart valve 10 in the closed position. In some embodiments, the clamping jaw 230 may be configured to cooperate with the elongate finger 222 to remove the portion 24 of the at least one valve leaflet 20 from the heart valve 10. Doing so may still separate (e.g., cut, resect, divide, incise, split, etc.) the at least one valve leaflet 20 into the plurality of discrete pieces 22, as seen in FIG. 10.

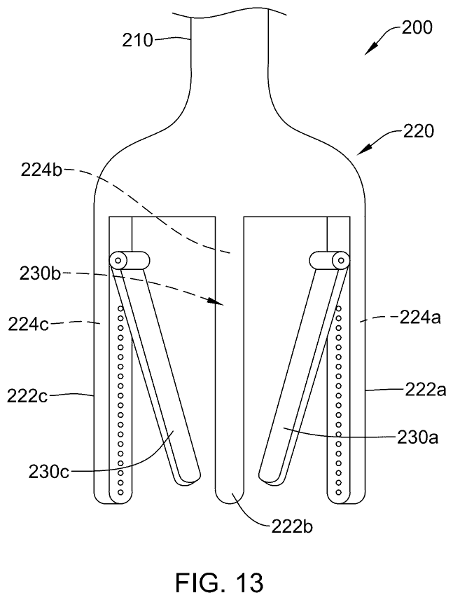

FIG. 13 illustrates selected aspects of an alternative configuration of the system 200 described herein. In some embodiments, the expandable structure 220 of the system 200 may comprise a first elongate finger 222a offset radially outward from the elongate shaft 210 in the expanded or offset configuration, the first elongate finger 222a having a lumen 224a extending therein and a first clamping jaw 230a disposed proximate a distal end of the first elongate finger 222a. In some embodiments, the expandable structure 220 of the system 200 may comprise a second elongate finger 222b offset radially outward from the elongate shaft 210 in the expanded or offset configuration, the second elongate finger 222b having a lumen 224b extending therein and a second clamping jaw 230b disposed proximate a distal end of the second elongate finger 222b. In some embodiments, the expandable structure 220 of the system 200 may comprise a third elongate finger 222c offset radially outward from the elongate shaft 210 in the expanded or offset configuration, the third elongate finger 222c having a lumen 224c extending therein and a third clamping jaw 230c disposed proximate a distal end of the third elongate finger 222c.

In some embodiments, the expandable structure 220 may be configured to separate (e.g., cut, resect, divide, incise, split, etc.) multiple valve leaflets (e.g., three valve leaflets) of the heart valve 10 into a plurality of discrete pieces 22 in a single operational step (e.g., without moving the expandable structure 220 from one leaflet to another, and/or without exchanging the system). In some embodiments, the expandable structure 220 may be configured to separate (e.g., cut, resect, divide, incise, split, etc.) each valve leaflet of the heart valve 10 into a plurality of discrete pieces 22 in a single operational step. Alternatively, in some embodiments, the expandable structure 220 may be configured to remove and/or excise a portion 24 of multiple valve leaflets (e.g., three valve leaflets), or each valve leaflet, of the heart valve 10 and/or separate those valve leaflets into a plurality of discrete pieces 22 in a single operational step (e.g., without moving the expandable structure 220 from one leaflet to another, and/or without exchanging the system). Other configurations, including combinations thereof, are also contemplated.

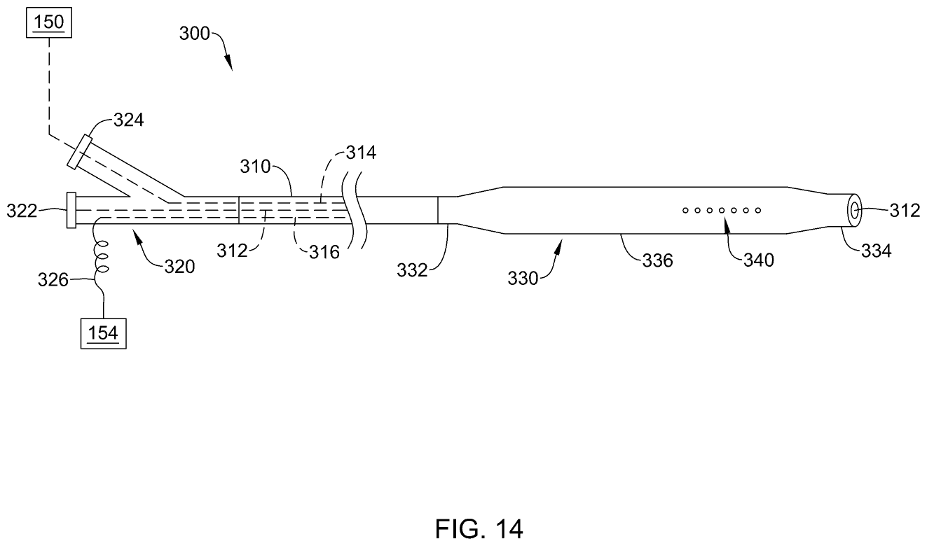

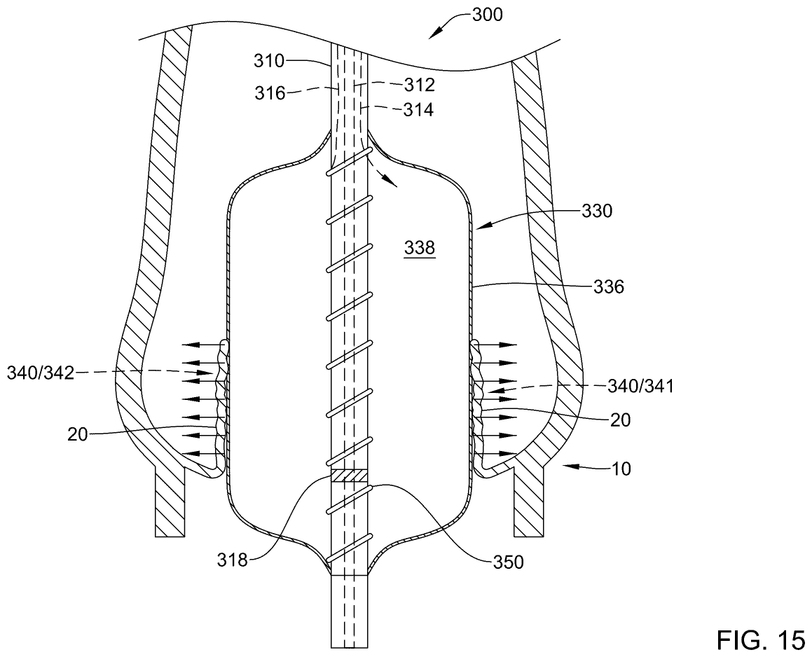

FIGS. 14-15 illustrate selected aspects of a system 300 for modifying at least one valve leaflet 20 of a heart valve 10 (e.g., an aortic valve, a replacement heart valve implant, etc.), shown in partial cutaway view in FIG. 15 for example. The system 300 may comprise an elongate shaft 310. In some embodiments, the elongate shaft 310 may comprise a proximal manifold 320 disposed at a proximal end of the elongate shaft 310. In some embodiments, the proximal manifold 320 may comprise a plurality of ports. In some embodiments, the proximal manifold 320 may comprise a guidewire port 322 in communication with a guidewire lumen 312 extending through the elongate shaft 310 and/or to a distal end of the elongate shaft 310. In some embodiments, the guidewire lumen 312 may open distally at a distalmost end of the elongate shaft 310. Other configurations are also contemplated.

In some embodiments, the system 300 may comprise an expandable balloon 330 disposed proximate the distal end of the elongate shaft 310. In some embodiments, the expandable balloon 330 may be disposed immediately adjacent the distal end of the elongate shaft 310. In some embodiments, the expandable balloon 330 may be configured to shift between a deflated configuration (e.g., FIG. 14) and an inflated configuration (e.g., FIG. 15) via an inflation fluid. In at least some embodiments, the expandable balloon 330 may comprise a proximal waist 332 fixedly attached to the elongate shaft 310, a distal waist 334 fixedly attached to the elongate shaft 310, and a body portion 336 extending between the proximal waist 332 and the distal waist 334. In some embodiments, the expandable balloon 330 may be formed from an elastic material. In some embodiments, the expandable balloon 330 may be formed from an inelastic material. In at least some embodiments, the expandable balloon 330 may be formed from a polymeric material. Other configurations are also contemplated.

In some embodiments, the proximal manifold 320 may comprise an inflation port 324 in fluid communication with an inflation lumen 314 extending from the inflation port 324 to the expandable balloon 330. In some embodiments, the inflation port 324 may be configured to be coupled to a source of pressurized fluid 150. The pressurized fluid may be inflation fluid. The inflation lumen 314 may be configured to transfer and/or transport pressurized fluid and/or inflation fluid from the source of pressurized fluid 150 to an interior 338 of the body portion 336 of the expandable balloon 330. In some embodiments, the pressurized fluid and/or the inflation fluid may be configured to receive and/or configured to accept an electrical charge.

In some embodiments, the proximal manifold 320 may comprise an electrical connection 326. The electrical connection 326 may be configured to be operatively coupled to an energy source, such as an RF generator 154. The proximal manifold 320 and/or the elongate shaft 310 may comprise a conductive wire 316 extending from the electrical connection 326 to the expandable balloon 330. In some embodiments, the conductive wire 316 may be disposed within the proximal manifold 320 and/or the elongate shaft 310. In some embodiments, the conductive wire 316 may extend along an outer surface of the proximal manifold 320 and/or the elongate shaft 310. Other configurations are also contemplated.

In some embodiments, the system 300 may comprise an electrode 350 disposed within the expandable balloon 330. The electrode 350 may be in communication with the inflation fluid within the interior 338 of the body portion 336 of the expandable balloon 330. In some embodiments, the electrode 350 may be a coiled wire disposed on and/or wrapped around the elongate shaft 310. In some embodiments, a portion of the electrode 350 may be disposed within and/or may be embedded within the elongate shaft 310. At least a portion of the electrode 350 may be exposed within the interior 338 of the body portion 336 of the expandable balloon 330 and/or in electrical contact with the inflation fluid disposed within the interior 338 of the body portion 336 of the expandable balloon 330. In at least some embodiments, the electrode 350 may be configured to energize the inflation fluid.

In some embodiments, the expandable balloon 330 and/or the body portion 336 of the expandable balloon 330 may comprise a plurality of small apertures 340 in fluid communication with the interior 338 of the body portion 336 of the expandable balloon 330, and/or in fluid communication with the inflation lumen 314, the inflation port 324, and/or the source of pressurized fluid 150. In some embodiments, the plurality of small apertures 340 may be disposed in and/or along a distal portion and/or a distal half of the body portion 336 of the expandable balloon 330.

In some embodiments, the plurality of small apertures 340 may comprise a first plurality of small apertures 341 in fluid communication with the interior 338 of the body portion 336 of the expandable balloon 330, and/or in fluid communication with the inflation lumen 314, the inflation port 324, and/or the source of pressurized fluid 150, a second plurality of small apertures 342 in fluid communication with the interior 338 of the body portion 336 of the expandable balloon 330, and/or in fluid communication with the inflation lumen 314, the inflation port 324, and/or the source of pressurized fluid 150, and/or a third plurality of small apertures (not shown) in fluid communication with the interior 338 of the body portion 336 of the expandable balloon 330, and/or in fluid communication with the inflation lumen 314, the inflation port 324, and/or the source of pressurized fluid 150.

In some embodiments, the plurality of small apertures 340, and/or the first plurality of small apertures 341, the second plurality of small apertures 342, and/or the third plurality of small apertures, may face radially outward and/or away from the expandable balloon 330. In some embodiments, the plurality of small apertures 340, and/or the first plurality of small apertures 341, the second plurality of small apertures 342, and/or the third plurality of small apertures, may face radially outward and/or away from a central longitudinal axis of the elongate shaft 310.

In at least some embodiments, in the inflation configuration, energized inflation fluid ejected through the plurality of small apertures 340, and/or the first plurality of small apertures 341, the second plurality of small apertures 342, and/or the third plurality of small apertures, may be configured to separate (e.g., cut, resect, divide, incise, split, etc.) the at least one valve leaflet 20 (e.g., FIG. 15) of the heart valve 10 into a plurality of discrete pieces 22 (e.g., FIG. 3). In some embodiments, in the inflation configuration, energized inflation fluid ejected through the plurality of small apertures 340, and/or the first plurality of small apertures 341, the second plurality of small apertures 342, and/or the third plurality of small apertures, may be configured to separate (e.g., cut, resect, divide, incise, split, etc.) each valve leaflet of the at least one valve leaflet 20 (e.g., FIG. 15) of the heart valve 10 into a plurality of discrete pieces 22 (e.g., FIG. 3).

In some embodiments, in the inflation configuration, the first plurality of small apertures 341 and/or energized inflation fluid ejected therethrough may be configured to separate (e.g., cut, resect, divide, incise, split, etc.) a first valve leaflet of the at least one valve leaflet 20 (e.g., FIG. 15) of the heart valve 10 into a first plurality of discrete pieces 22 (e.g., FIG. 3). In some embodiments, in the inflation configuration, the second plurality of small apertures 342 and/or energized inflation fluid ejected therethrough may be configured to separate (e.g., cut, resect, divide, incise, split, etc.) a second valve leaflet of the at least one valve leaflet 20 (e.g., FIG. 15) of the heart valve 10 into a second plurality of discrete pieces 22 (e.g., FIG. 3). In some embodiments, in the inflation configuration, the third plurality of small apertures and/or energized inflation fluid ejected therethrough may be configured to separate (e.g., cut, resect, divide, incise, split, etc.) a third valve leaflet of the at least one valve leaflet 20 (e.g., FIG. 15) of the heart valve 10 into a third plurality of discrete pieces 22 (e.g., FIG. 3). Other configurations are also contemplated.

In some embodiments, the plurality of small apertures 340, and/or the first plurality of small apertures 341, the second plurality of small apertures 342, and/or the third plurality of small apertures, may be arranged in a substantially straight line or a linear pattern. In some embodiments, the plurality of small apertures 340, and/or the first plurality of small apertures 341, the second plurality of small apertures 342, and/or the third plurality of small apertures, may be arranged longitudinally and/or axially. In some embodiments, the plurality of small apertures 340, and/or the first plurality of small apertures 341, the second plurality of small apertures 342, and/or the third plurality of small apertures, may be arranged parallel with a central longitudinal axis of the elongate shaft 310. In some embodiments, the plurality of small apertures 340, and/or the first plurality of small apertures 341, the second plurality of small apertures 342, and/or the third plurality of small apertures, may be arranged in a non-linear pattern (e.g., a zig-zag shape, a curve, etc.). In some embodiments, the plurality of small apertures 340, and/or the first plurality of small apertures 341, the second plurality of small apertures 342, and/or the third plurality of small apertures, may be arranged in various patterns and/or configurations (e.g., different geometries, etc.).

In some embodiments, the at least one valve leaflet 20, and/or each leaflet of the at least one valve leaflet 20, may be cut and/or slit in a generally straight line. In embodiments, a portion or portions of the at least one valve leaflet 20, and/or each leaflet of the at least one valve leaflet 20, may be excised or removed entirely. Other configurations are also contemplated.

In some embodiments, the system 300 may be monopolar system. In some embodiments, the system 300 may be a bipolar system. In some embodiments, the electrode 350 may comprise a first pole. In some embodiments, the patient's body and/or tissue may comprise a second pole. In some embodiments, a second pole may be disposed on an outer surface of the expandable balloon 330 and/or the elongate shaft 310. The second pole may be electrically spaced apart from and/or isolated from the first pole such that the inflation fluid conducts electrical energy from the first pole to the second pole.

Energized inflation fluid, directed through the plurality of small apertures 340, and/or the first plurality of small apertures 341, the second plurality of small apertures 342, and/or the third plurality of small apertures, may serve to at least partially ablate a portion of the at least one valve leaflet 20, thereby enhancing and/or speeding up separation of the at least one valve leaflet 20 (e.g., FIG. 15) of the heart valve 10 into the plurality of discrete pieces 22 (e.g., FIG. 3). Energized inflation fluid, directed through the plurality of small apertures 340, and/or the first plurality of small apertures 341, the second plurality of small apertures 342, and/or the third plurality of small apertures, may serve to at least partially ablate a portion of each leaflet of the at least one valve leaflet 20, thereby enhancing and/or speeding up separation of each leaflet of the at least one valve leaflet 20 (e.g., FIG. 15) of the heart valve 10 into the plurality of discrete pieces 22 (e.g., FIG. 3). Additionally, the inflation fluid may provide cooling to the tissue (e.g., the at least one valve leaflet 20) being ablated.

In some embodiments, the system 300 and/or the elongate shaft 310 may comprise a marker element 318 configured to align the plurality of small apertures 340, and/or the first plurality of small apertures 341, the second plurality of small apertures 342, and/or the third plurality of small apertures, with the at least one valve leaflet 20 of the heart valve 10 under fluoroscopy. During use, the heart valve 10 may be imaged using convention means and fluoroscopy may be used to align the marker element 318 with the annulus of the heart valve 10 such that the plurality of small apertures 340, and/or the first plurality of small apertures 341, the second plurality of small apertures 342, and/or the third plurality of small apertures, is aligned with the at least one valve leaflet 20 of the heart valve 10. Other configurations and/or means of aligning and/or orienting the system 300 are also contemplated.

In some embodiments, the system 300 may comprise a delivery catheter (not shown), similar to the delivery catheter 102 shown in FIGS. 2 and 4. In some embodiments, the elongate shaft 310 and the expandable balloon 330 may be axially slidable within a lumen of the delivery catheter. In some embodiments, the delivery catheter may comprise an aspiration function and/or capability. In some embodiments, the system 300 may comprise a separate aspiration catheter delivered and/or extending alongside the elongate shaft 310. Other configurations are also contemplated. In some embodiments, the system 300 may be devoid of the delivery catheter.

The materials that can be used for the various components of the system and the various elements thereof disclosed herein may include those commonly associated with medical devices. For simplicity purposes, the following discussion refers to the system. However, this is not intended to limit the devices, components, and methods described herein, as the discussion may be applied to other elements, members, components, or devices disclosed herein, such as, but not limited to, the elongate shaft, the expandable balloon, the expandable structure, etc. and/or elements or components thereof.

In some embodiments, the system and/or components thereof may be made from a metal, metal alloy, polymer, a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM; for example, DELRIN®), polyether block ester, polyurethane, polypropylene (PP), polyvinylchloride (PVC), polyether-ester (for example, ARNITEL®), ether or ester based copolymers (for example, butylene/poly(alkylene ether) phthalate and/or other polyester elastomers such as HYTREL®), polyamide (for example, DURETHAN® or CRISTAMID®), elastomeric polyamides, block polyamide/ethers, polyether block amide (PEBA; for example, PEBAX®), ethylene vinyl acetate copolymers (EVA), silicones, polyethylene (PE), MARLEX® high-density polyethylene, MARLEX® low-density polyethylene, linear low density polyethylene (for example, REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, Elast-Eon® or ChronoSil®), biocompatible polymers, other suitable materials, or mixtures, combinations, copolymers thereof, polymer/metal composites, and the like. In some embodiments, the system and/or components thereof can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some embodiments, portions or all of the system and/or components thereof may also be doped with, made of, or otherwise include a radiopaque material. Radiopaque materials are understood to be materials capable of producing a relatively bright image on a fluoroscopy screen or another imaging technique (e.g., ultrasound, etc.) during a medical procedure. This relatively bright image aids the user of the system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some embodiments, the system and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); immunosuppressants (such as the “olimus” family of drugs, rapamycin analogues, macrolide antibiotics, biolimus, everolimus, zotarolimus, temsirolimus, picrolimus, novolimus, myolimus, tacrolimus, sirolimus, pimecrolimus, etc.); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A system for modifying at least one valve leaflet of a heart valve, comprising:
an elongate shaft;
an expandable balloon disposed proximate a distal end of the elongate shaft, wherein the expandable balloon is configured to shift between a deflated configuration and an inflated configuration; and
an expandable structure disposed radially outward of the expandable balloon, wherein the expandable structure is configured to shift between a collapsed configuration and an expanded configuration;
wherein in the expanded configuration, the expandable structure is configured to separate the at least one valve leaflet into a plurality of discrete pieces.

2. The system of claim 1, wherein the expandable structure comprises at least one elongate finger having a lumen extending therein;
wherein a distal portion of each finger of the at least one elongate finger comprises a plurality of apertures formed therein in fluid communication with the lumen.

3. The system of claim 2, wherein the plurality of apertures faces radially outward from the expandable balloon.

4. The system of claim 2, wherein the lumen is in fluid communication with a source of pressurized fluid.

5. The system of claim 4, wherein pressurized fluid ejected from the plurality of apertures is configured to separate the at least one valve leaflet into the plurality of discrete pieces.

6. The system of claim 2, wherein the lumen is in fluid communication with a vacuum source.

7. The system of claim 6, wherein a vacuum applied to the plurality of apertures is configured to draw a portion of the at least one valve leaflet into their respective finger to separate the at least one valve leaflet into the plurality of discrete pieces.

8. A system for modifying at least one valve leaflet of a heart valve, comprising:
an elongate shaft; and
an expandable structure disposed at a distal end of the elongate shaft, the expandable structure comprising an elongate finger having a lumen extending therein and a clamping jaw disposed proximate a distal end of the elongate finger;
wherein the expandable structure is configured to shift between a collapsed configuration and an expanded configuration;
wherein in the expanded configuration, the elongate finger is offset radially outward from the elongate shaft and is configured to separate the at least one valve leaflet into a plurality of discrete pieces.

9. The system of claim 8, wherein the clamping jaw is pivotably coupled to the elongate finger and configured to open radially inward toward a central longitudinal axis of the elongate shaft.

10. The system of claim 8, wherein the elongate finger comprises a plurality of apertures formed therein in fluid communication with the lumen.

11. The system of claim 10, wherein the lumen is in fluid communication with a source of pressurized fluid.

12. The system of claim 10, wherein the lumen is in fluid communication with a vacuum source.

13. The system of claim 10, wherein the plurality of apertures faces toward the clamping jaw.

14. The system of claim 13, wherein the plurality of apertures is arranged linearly along the elongate finger.

15. The system of claim 13, wherein the plurality of apertures is arranged along a perimeter of the elongate finger.

16. The system of claim 13, wherein the clamping jaw is configured to cooperate with the elongate finger to grasp a portion of the at least one valve leaflet and remove the portion of at least one valve leaflet from the heart valve.

17. The system of claim 13, wherein the expandable structure further comprises:
a second elongate finger offset radially outward from the elongate shaft, the second elongate finger having a lumen extending therein and a second clamping jaw disposed proximate a distal end of the second elongate finger; and
a third elongate finger offset radially outward from the elongate shaft, the third elongate finger having a lumen extending therein and a third clamping jaw disposed proximate a distal end of the third elongate finger.

18. A system for modifying at least one valve leaflet of a heart valve, comprising:
an elongate shaft;
an expandable balloon disposed proximate a distal end of the elongate shaft, wherein the expandable balloon is configured to shift between a deflated configuration and an inflated configuration via an inflation fluid; and
an electrode disposed within the expandable balloon and in communication with the inflation fluid, wherein the electrode is configured to energize the inflation fluid;
wherein the expandable balloon comprises a proximal waist, a distal waist, and a body portion extending between the proximal waist and the distal waist;
wherein the body portion of the expandable balloon comprises a plurality of small apertures in fluid communication with an interior of the body portion of the expandable balloon;
wherein in the inflated configuration, energized inflation fluid ejected through the plurality of small apertures is configured to separate the at least one valve leaflet into a plurality of discrete pieces.

19. The system of claim 18, wherein the elongate shaft comprises a marker element configured to align the plurality of small apertures with the at least one valve leaflet under fluoroscopy.

20. The system of claim 18, wherein the plurality of small apertures comprises a first plurality of small apertures configured to separate a first valve leaflet into a first plurality of discrete pieces, and a second plurality of small apertures configured to separate a second valve leaflet into a second plurality of discrete pieces.
